(19) United States
(12) Patent Application Publication
(10) Pub. No.: US 2009/0018456 A1
(43) Pub. Date: Jan. 15, 2009

(54) Display storage apparatus capable of detecting a pulse

(76) Inventor: Chin-Yeh Hung, Taoyuan City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/822,881

(22) Filed: Jul. 11, 2007

(51) Int. Cl. A61B 5/0402

(52) U.S. Cl. 600/509

(57) ABSTRACT

A display storage apparatus capable of detecting a pulse made in form of a pulse MP3/4 or a wristwatch and comprising: a) a casing having an LCD screen disposed at the front side thereof; b) a heartbeat detector unit having a plurality of electrode patches extended out from the casing and directly attached onto a human chest for detecting a pulse, the signal being transmitted in a way of wire or wireless transmission; c) a voltage sensor unit installed in the casing for sending a voltage change frequency; d) a digital signal processor for receiving a signal transmitted from the voltage sensor unit, and cumulating the signals to obtain a user's pulse (or heartbeat) value; e) a flash memory having a plurality of partitioned memory blocks with at least one memory block for storing an Auto Run program; f) a USB interface disposed on a lateral side of the casing, and electrically coupled to the digital signal processor, and if the USB interface is connected to a personal computer, the Auto Run program stored in the flash memory automatically stores the measured data of a user's pulses accumulated in a certain period of time into an operating system of the personal computer and further analyzes and record the user's physiological conditions. In this way, the apparatus can be used for listening to music anytime, displaying the current time, analyzing and evaluating a user's exercise and physiological conditions.

### BACKGROUND OF THE INVENTION

1. Field of the Invention

The invention relates to a display storage apparatus capable of detecting a pulse, and more particularly to a display storage apparatus capable of measuring a heartbeat, cumulating and recording the measured data in a flash memory, using an AuTo Run program and a USB interface for quickly and conveniently transmitting the measured data to a personal computer for further analyses and evaluations.

2. Description of the Related Art

Movie Picture Experts Group Layer 3/4 (MP3/4) 1 is one of the layers of the MPEG-1, and whose original purpose of development is to reduce the negative load consumed in an information medium transmission, and MP3/4 sound data is one of the sound wave data that compresses the original sound wave data into the format of MP3/4 1. With a special compression method, the wave data of approximately 50 MB to 60 MB can be compressed into approximately 4 MB (with a difference of almost 10 times), and the sound quality is maintained at a level equivalent to the sound effect of a music CD, and thus a MP3/4 music CD can store the data of more than 10 music CDs. For instance, if a music CD album can play music for 60 minutes, and a MP3/4 music CD can play music for more than 10 hours, the MP3/4 music CD acts like a small music jukebox and provides convenience and portability to users, and thus the MP3/4 music CD becomes a fashion now. In addition, the MP4 belongs to a MPEG-2 AAC technical level, since its sound quality is better and its compression ratio (15:1˜20:1) is larger, and thus it becomes popular gradually.

At present, increasingly more people (such as walkers or joggers) listen to a MP3/4 player while doing exercises, and our heartbeat value varies significantly before and after the exercise, and the measurement of these variables during the exercise are very important to maximize the benefit of the exercise. The exercise needs to increase our heartbeat rate up to 60% of the maximum rate for the exerciser's best benefit, but it will do us harm, if the exercise heartbeat rate is increased to the maximum rate. An accurate and convenient-to-measure heartbeat meter is an indispensable instrument for fitness exercises.

A variety of traditional fitness exercise type heartbeat measuring devices such as the pulse meters disclosed in U.S. Pat. Nos. 4,409,983, 4,224,948, 4,120,269 and 5,807,267 are available in the market. Some of the traditional pulse meters use an infrared sensor for detecting and measuring the pulse, and some use exhaust sensors, piezo pressure sensors or optical sensors for the detection and measurement. The pulse meters adopting the infrared sensor may be affected by ambient environmental conditions, and thus the precision is relatively low.

In addition, U.S. Pat. No. 5,807,267 installs a sensor at the bottom of a wrist band for detecting and measuring the heartbeat value of a radial artery, but the sensor assembly of a piezo pressure sensor or an optical sensors attached onto the radial artery for the detection and measurement may be affected by the ambient environmental conditions, and a precise position of its aligned position substantially affects the measurement. Medical professionals may be able to obtain an accurate measurement by using the aforementioned instrument, but an athlete or a general consumer may find it difficult to achieve a precise alignment. Furthermore, the traditional pulse detectors do not come with the recording and analyzing functions, and they cannot be connected to a personal computer, and thus the traditional pulse detectors cannot record and provide users a variation of heartbeat values within a time period such as a few days, a week or a month, and they cannot be used for analyzing and evaluating the physical conditions of users. These traditional pulse detectors definitely require further improvements.

### SUMMARY OF THE INVENTION

It is a primary object of the invention to provide a display storage apparatus capable of detecting a pulse, wherein a MP3/4 or a wrist watch is skillfully integrated with a pulse detector for measuring a heartbeat (pulse) value in addition to the function of listening to music anytime and displaying the current time.

It is another object of the present invention to provide a display storage apparatus capable of detecting a pulse, wherein the detected heartbeat values are accumulated and stored, and a built-in Auto Run program and a USB interface are provided to quickly connect with a personal computer anytime for automatically sending a record of heartbeat values with a certain specific time to the personal computer for a comprehensive analysis and evaluation, and providing detailed personal health information of a user.

It is a further object of the present invention is to send the heartbeat record stored in the personal computer to a doctor's personal computer at a remote end through the Internet and a platform server for further evaluations and bidirectional communications.

In order to achieve the above-mentioned objects, a display storage apparatus capable of detecting a pulse includes:

a) a casing having an LCD screen disposed at the front side thereof and connected to an external signal insert hole;

b) a heartbeat detector unit having a plurality of electrode patches extended out from the casing, the heartbeat detector unit directly attached onto a human chest for detecting a pulse and returning a measured heartbeat signal into the casing;

c) a voltage sensor unit installed in the casing, and having three inductors and a filter waveform-shaping amplification circuit, for sending a voltage change frequency measured by the heartbeat detector unit to the filter waveform-shaping amplification circuit to obtain a signal, after electric charges are filtered by the inductors;

d) a digital signal processor for receiving a signal transmitted from the voltage sensor unit, and cumulating the signals to obtain a user's pulse (or heartbeat) value;

e) a flash memory having a plurality of partitioned memory blocks with at least one memory block for storing an Auto Run program and other memory blocks serving as general memories, and the flash memory being provided for storing audio data, and producing an audio signal after the audio data is decoded by the digital signal processor;

f) a power supply unit installed in the casing, for supplying the required electric power to the foregoing elements;

g) a plurality of input keys disposed on a surface of the casing, and provided for users to operate and transmit an instruction to the digital signal processor; and;

h) a USB interface disposed on a lateral side of the casing, and electrically coupled to the digital signal processor, and if the USB interface is connected to a personal computer, the Auto Run program stored in the flash memory automatically stores the measured data of a user's pulses accumulated in a certain period of time into an operating system of the personal computer and further analyzes and record the user's physiological conditions.

### BRIEF DESCRIPTION OF THE FIGURES

The accomplishment of this and other objects of the invention will become apparent from the following descriptions and its accompanying figures of which:

FIG. 1 is a perspective view of a first preferred embodiment of the present invention;

FIG. 2 is a schematic view of a first preferred embodiment and a USB connected in accordance with the present invention;

FIG. 3 is a schematic view of measuring a heartbeat in accordance with a first preferred embodiment of the present invention;

FIG. 4 is a circuit block diagram of a first preferred embodiment of the present invention;

FIG. 5 is a schematic view of measuring a heartbeat in accordance with a second preferred embodiment of the present invention;

FIG. 6 is a circuit block diagram of a second preferred embodiment of the present invention;

FIG. 7 is a schematic view of the present invention being connected to a computer in accordance with the present invention;

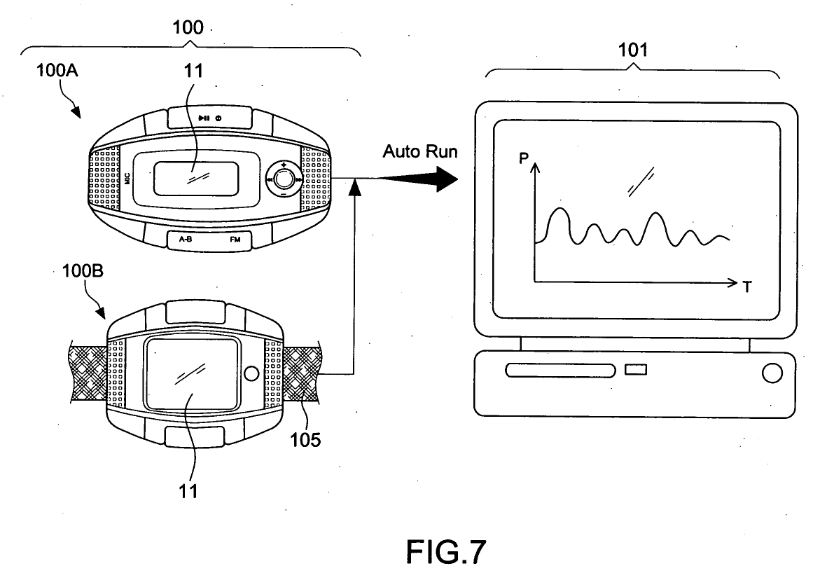

FIG. 8 is a schematic view of an application of the present invention; and

FIG. 9 is a schematic view of another application of the present invention.

### DETAILED DESCRIPTION OF THE PREFERRED EMBODIMENT

A display storage apparatus capable of detecting a pulse 100 in accordance with the present invention can be made in a form of a pulse MP3/4 player 100A, and also in a form of a wrist watch 100B, and they are described as follows:

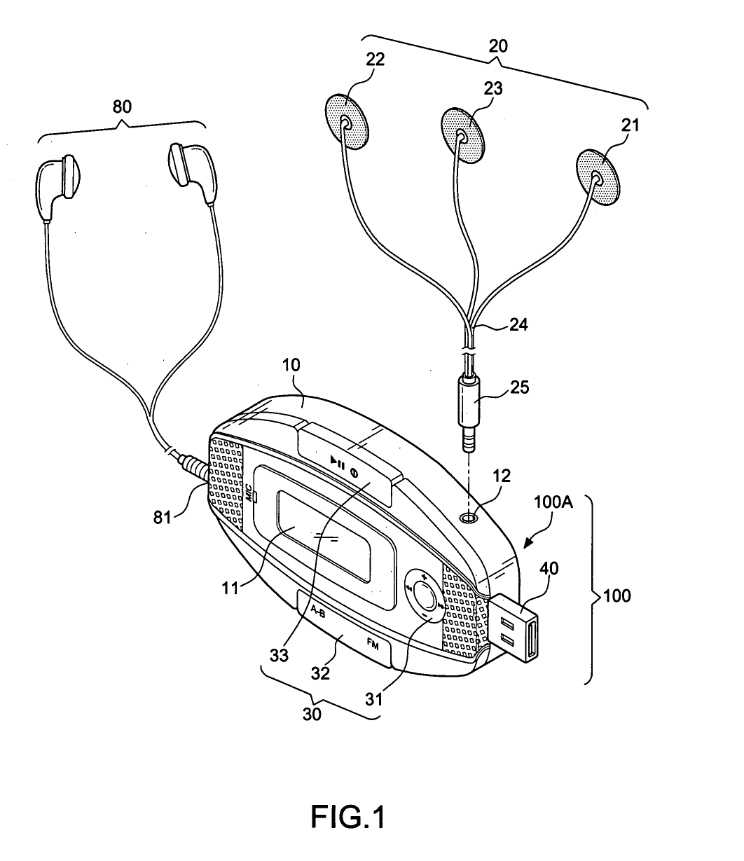

Referring to FIGS. 1 to 4, the pulse MP3/4 player 100A in accordance with a first preferred embodiment of the present invention comprises:

a casing 10, with the shape and the size of a general MP3/4 player, and having an CD screen 11 disposed at a front side of the casing 10 and connected to an external signal insert hole 12;

a heartbeat detector unit 20, externally coupled to the insert hole 12, and comprised of at least three electrode patches respectively an anode patch 21, a cathode patch 22 and a common electrode patch 23, and the three electrode patches 21, 22, 23 being coupled to three conducting wires 24, and a terminal of the conducting wire 24 being coupled to a coaxial signal plug 25, and connectable to the insert hole 12; and

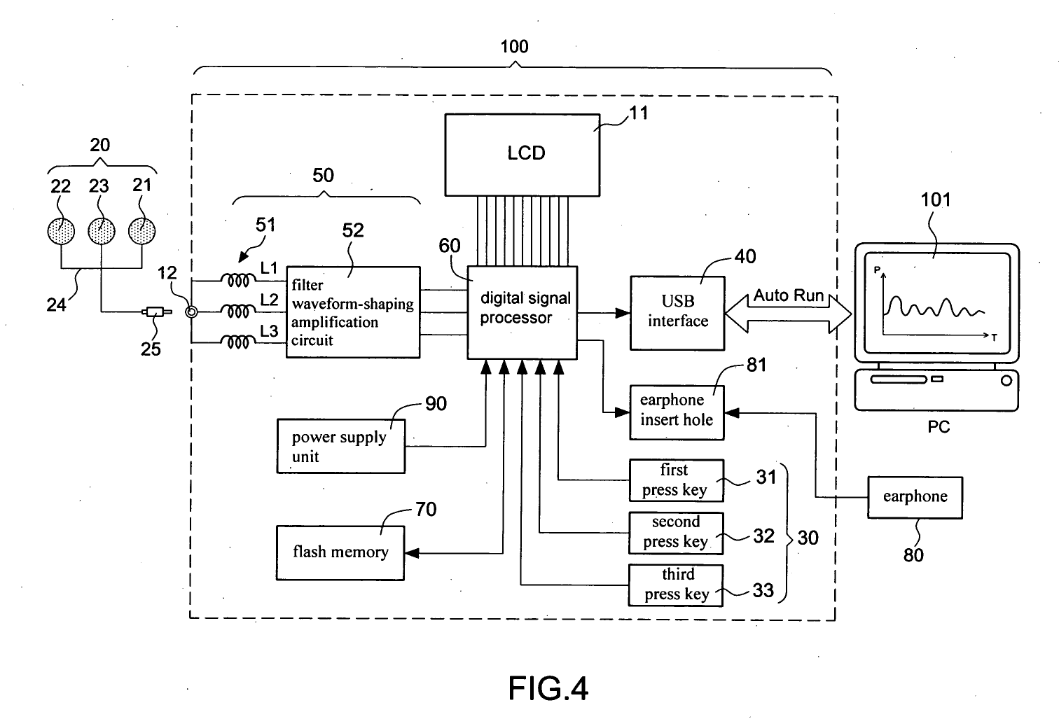

a voltage sensor unit 50, installed in the casing 10, for detecting a voltage frequency change caused by the blood circulation of a human body human, and having three inductors 51 and a filter waveform-shaping amplification circuit 52 as shown in FIG. 4, wherein the three inductors 51 are electrically coupled to the insert holes 12 for detecting a voltage change frequency measured by the heartbeat detector unit 20, and the voltage change frequency being sent to the filter waveform-shaping amplification circuit 52 to obtain a signal after electric charges are filtered by the inductor 51.

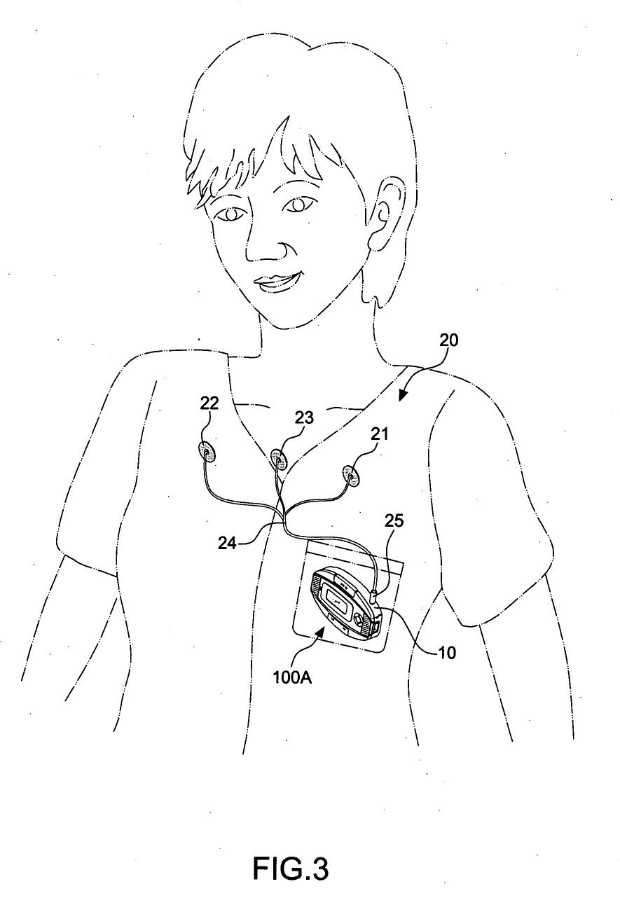

The present invention detects a heartbeat as shown in FIG. 3, the MP3/4 casing 10 is placed at an appropriate position of the user's body such as a pocket or a belt, and then the three electrode patches 21, 22 23 of heartbeat detector unit 20 are attached onto a human chest, and the three electrode patches 21, 22, 23 are made of a metal sheet or an electrically conducting rubber with an excellent conductivity and adhesiveness at the contact surface with the human chest. The electrode patches 21, 22, 23 can be attached onto a chest easily without the risk of being slid. The anode and cathode patches 21, 22 are preferably and respectively attached on left and right sides of the front of the chest cavity, and a voltage will be produced by the circulation of human blood. Different heartbeat rates give different voltage frequency, and the farther from the human blood, the more significant is the change of voltage frequency. The present invention attaches different anode and cathode patches 21, 22 to the user's left and right chest respectively to measure the heartbeat value accurately.

Referring to FIG. 4, a digital signal processor (DSP) 60 comes with a MCU of a most significant bit high-speed operation and data pointer displacement instruction, and has a programmable digital signal processing capability for the voice, communication, image processing and data control applications. In this preferred embodiment, the digital signal processor 60 can execute a program stored in a flash memory 70 for controlling, playing and operating a decoded MP3/4 file or video comprised file to generate the corresponding audio signals which are used for the general MP3/4 playback function. Further, the digital signal processor 60 receives a signal transmitted from the foregoing voltage sensor unit 50, and cumulates the signals to obtain a user's heartbeat value pulse, and of course, the heartbeat values can be displayed on the LCD screen 20.

Since the present pulse detector such as the aforementioned pulse meter can detect the heartbeat value only, but cannot record and analyze the heartbeat value. Therefore, users cannot know about the situation of their pulse change within a week or a month and it is difficult for users to evaluate their physiological and health conditions.

To overcome the foregoing shortcoming of the prior art, the present invention uses the flash memory 70 as memory unit, and has a plurality partitioned memory blocks, with at least one block for storing an Auto Run program, and other blocks for the use of a general memory, and the flash memory 70 is provided for storing audio data, and then the digital signal processor 60 decodes the audio data to produce an audio signal.

Referring to FIG. 1, the present invention further comprises: an earphone insert hole 81 disposed on a lateral side of the casing 10 for externally connecting an earphone 80, and converting the audio signal outputted by the digital signal processor 60 into a sound. A plurality of input keys 30 are disposed on a surface of the casing 10 and provided for users to operate and transmit an instruction to the digital signal processor 60; for example, a first press key 31 is used for the ON/OFF/Play/Pause functions, and a second press key 32 is used as the direction keys, and a third press key is used for the Record/FM function, but these press keys are not limited to such particular functions and can be changed according to the design requirements.

Referring to FIG. 4, a power supply unit 90 is installed in the casing 10 for supplying the required electric power for each component, and the power supply unit 90 can be an AAA battery or a lithium battery, etc.

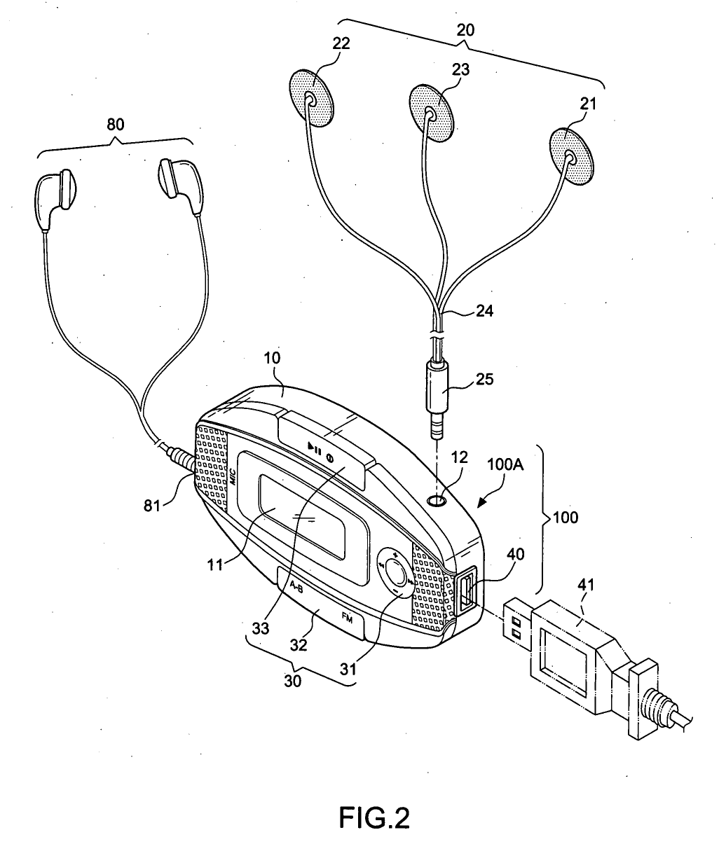

A USB interface 40 is installed on a lateral side of the casing 10 as shown in FIG. 1, and the USB interface 40 can be in a form of a male connector and concavely disposed on a lateral side of the casing 10. Of course, the USB interface 40 can be in a form of a female connector and concavely disposed in the casing 10 as shown in FIG. 2 and the USB interface 40 uses a USB cable 41 for the connection.

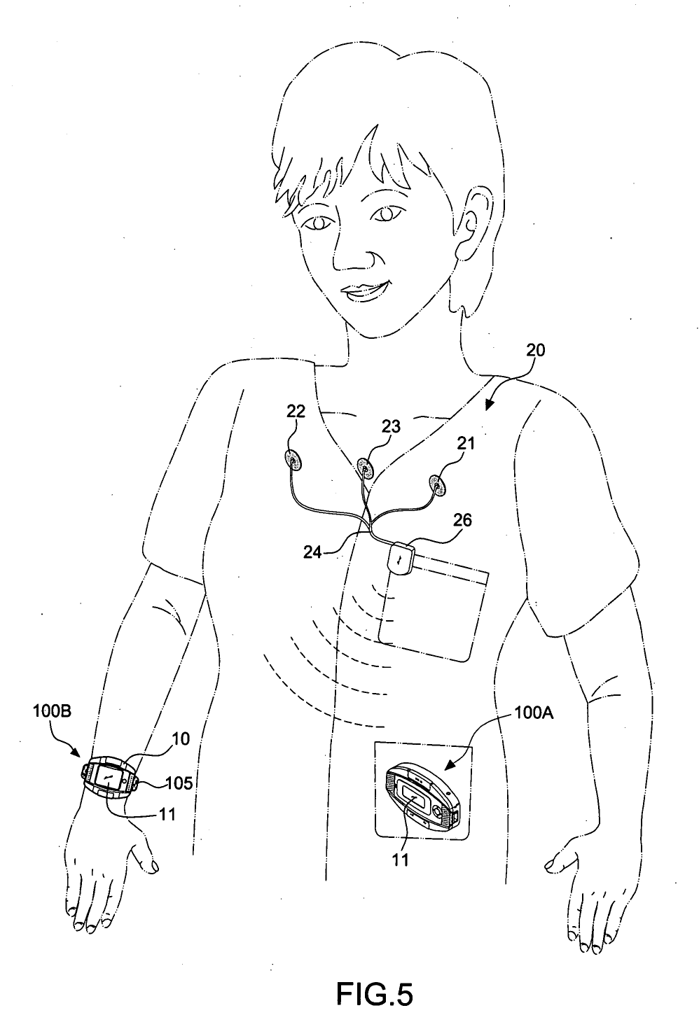

In FIG. 4, the USB interface 40 is electrically coupled to the digital signal processor 60, and when the USB interface 40 is connected to a personal computer 101, an Auto Run program stored in the flash memory 70 can transmit the user's measured pulses accumulated in a specific time period such as a week, a month or a year to the personal computer 101 as shown in FIG. 5, and the measured pulses can be applied to the original software, hardware and operating system of the personal computer 101. When the pulse MP3/4 player 100A of the invention is connected to the personal computer 101, the measured pulses can be transmitted automatically to provide a very convenient application, so that users can obtain the pulse change within a predetermined time period, and use a graphical method for the analysis and evaluation to let users know about their physical conditions much better.

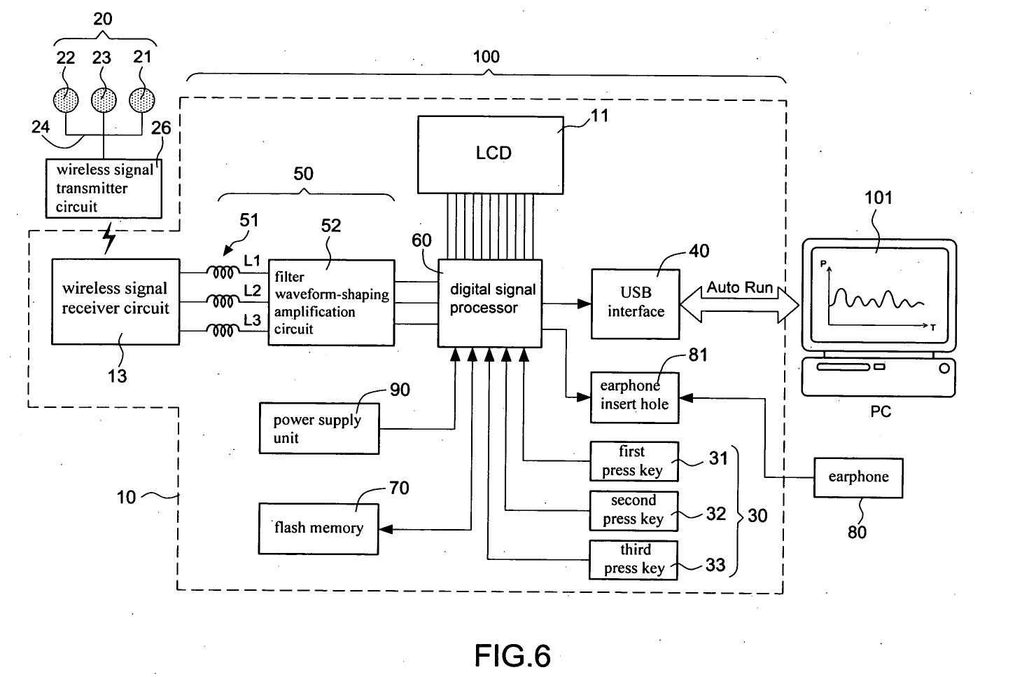

Referring to FIGS. 5 to 6 for a schematic view and a circuit block diagram of a second preferred embodiment of the present invention, the same elements with the first preferred embodiment use the same numerals for the illustration purpose, and the difference with the first preferred embodiment resides on that the heartbeat detector unit 20 of the first preferred embodiment uses a cable transmission method to send the measured heartbeat signal back to the casing 10, but the second preferred embodiment of the invention uses a wireless transmission method to send the measured heartbeat signal to the casing 10, and the terminal of each conducting wire 24 of a plurality of electrode patches 21, 22, 23 is connected to a wireless signal transmitter circuit 26 for transmitting the signal, and the measured heartbeat signal is received by the wireless signal receiver circuit 13 in the casing 10. Similar to the previous preferred embodiment, the voltage sensor unit 50 in the casing 10 can obtain the voltage change frequency signal measured by the heartbeat detector unit 20.

Referring to FIG. 6 for the wireless transmission mode, the display storage apparatus 100 can be made in a form of a pulse MP3/4 player 100A as shown in FIG. 5 and in a form of a wrist watch 100B which has a wrist band 105 disposed on both sides of the casing 10 and can be worn on a user's hand. The internal structure or circuit of the wrist watch 100B is substantially the same as that of the pulse MP3/4, and its surface also has an LCD screen 11. Therefore, a user can use the heartbeat detector unit 20 to detect the heartbeat value and display the heartbeat value from the wrist watch 100B while doing exercises.

The technical measures of the invention as described above include the following advantages and effects:

1. In the present invention, the heartbeat detector unit 20 is extended out from the casing 10 and attached directly onto a user's chest for measuring a heartbeat, and thus the measurement is relatively more accurate.

2. In the present invention, the heartbeat detector unit 20 can be connected to the display storage apparatus 100 quickly via a cable or wireless transmission, and thus users can listen to music or display time while doing exercises, and also can detect and measure the heartbeat value concurrently.

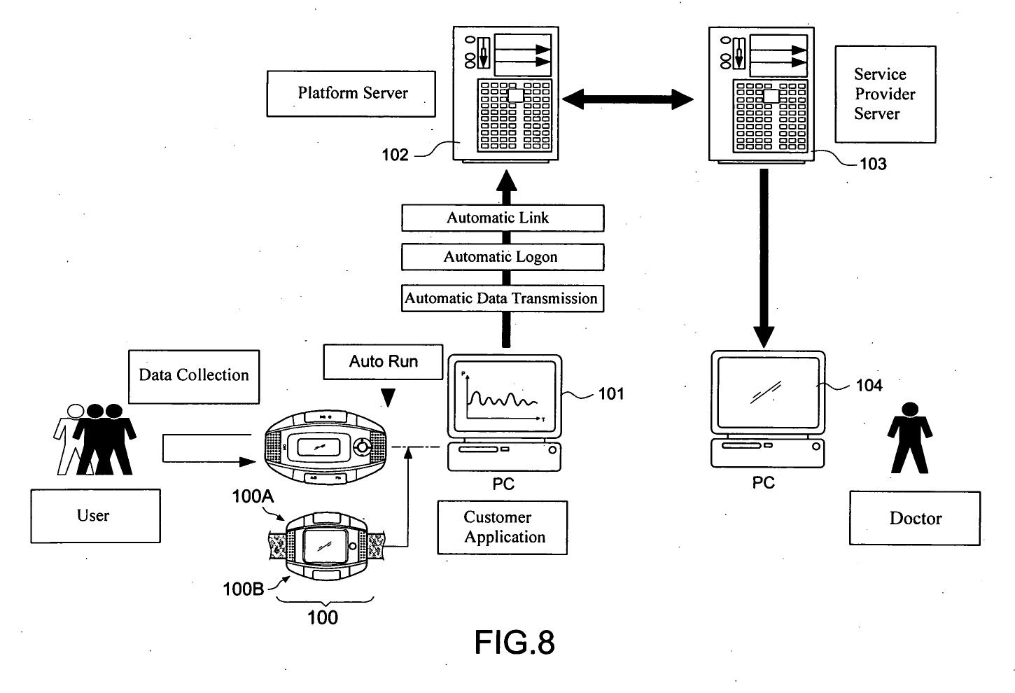

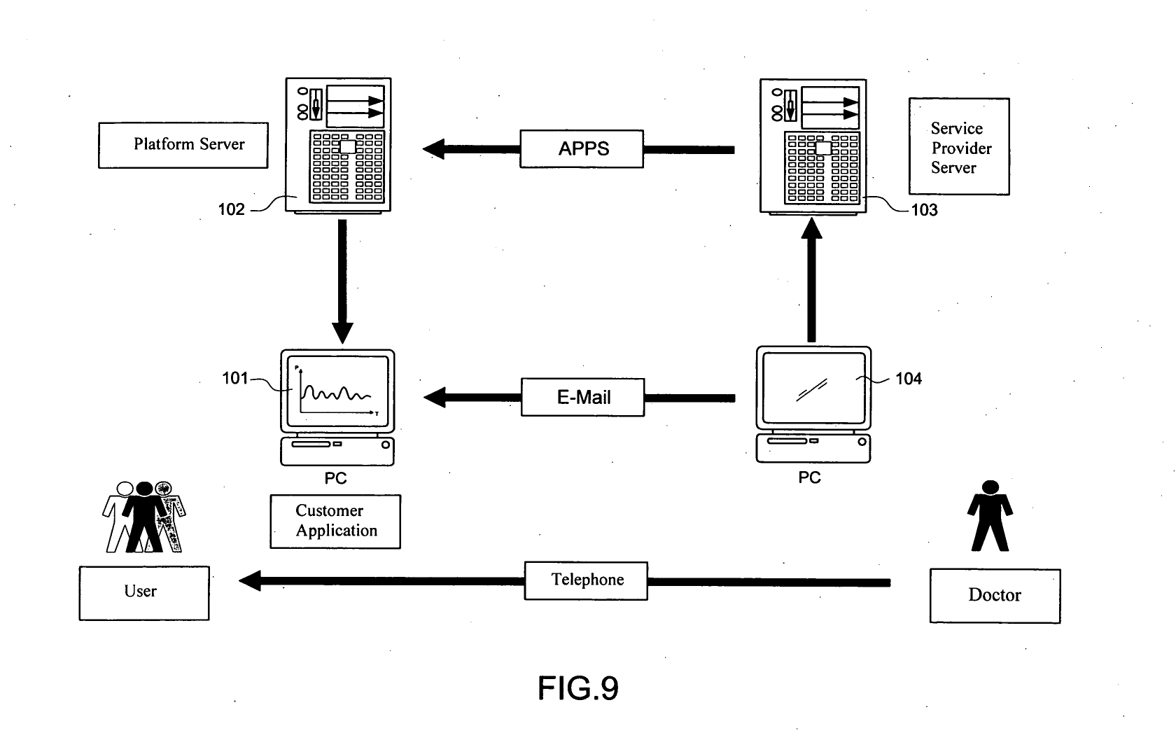

3. The present invention uses an Auto Run program stored in a flash memory 70 to immediately display all heartbeat values measured within a specific time period on a personal computer 101, such that when the display storage apparatus 100 composed of a pulse MP3/4 player 100A or a wrist watch 100B is connected to the personal computer 101, users can know about their physiological conditions. In FIG. 8, the collected user data is transmitted automatically, logged on and linked to a platform server 102 by a personal computer 101 and also linked to a service provider server 103, such that a doctor at a remote site can obtain the user's physiological information from the personal computer 64 for periodic tracking and suggestions. In FIG. 9, the doctor can send the analysis and evaluation and inform the user through telephone or E-mail or upload the data to the service provider server 63, and users can know about the doctor's analysis reports or suggestions through the platform server 62 from their personal computer 61 conveniently

Many changes and modifications in the above-described embodiments of the invention can, of course, be carried out without departing from the scope thereof. Accordingly, to promote the progress in science and the useful arts, the invention is disclosed and is intended to be limited only by the scope of the appended claims.

What is claimed is:

1. A display storage apparatus capable of detecting a pulse, comprising:
a) a casing having an LCD screen disposed at the front side thereof;
b) a heartbeat detector unit having a plurality of electrode patches extended out from the casing, the heartbeat detector unit directly attached onto a human chest for detecting a pulse and returning a measured heartbeat signal into the casing;
c) a voltage sensor unit installed in the casing, and having three inductors and a filter waveform-shaping amplification circuit, for sending a voltage change frequency measured by the heartbeat detector unit to the filter waveform-shaping amplification circuit to obtain a signal, after electric charges are filtered by the inductors;
d) a digital signal processor for receiving a signal transmitted from the voltage sensor unit, and cumulating the signals to obtain a user's pulse (or heartbeat) value;
e) a flash memory having a plurality of partitioned memory blocks with at least one memory block for storing an Auto Run program and other memory blocks serving as general memories, and the flash memory being provided for storing audio data, and producing an audio signal after the audio data is decoded by the digital signal processor;
f) a power supply unit installed in the casing, for supplying the required electric power to the foregoing elements;
g) a plurality of input keys disposed on a surface of the casing, and provided for users to operate and transmit an instruction to the digital signal processor; and;
h) a USB interface disposed on a lateral side of the casing, and electrically coupled to the digital signal processor, and if the USB interface is connected to a personal computer, the Auto Run program stored in the flash memory automatically stores the measured data of a user's pulses accumulated in a certain period of time into an operating system of the personal computer and further analyzes and record the user's physiological conditions.

2. The display storage apparatus capable of detecting a pulse as recited in claim 1 wherein the casing is made in a form of a MP3/4 having an earphone insert hole disposed on a side of the casing.

3. The display storage apparatus capable of detecting a pulse as recited in claim 1 wherein the casing is made in a form of a wrist watch having a wrist band disposed on a side of the casing.

4. The display storage apparatus capable of detecting a pulse as recited in claim 1 wherein the heartbeat detector unit sends a measured heartbeat signal back to the casing by a cable transmission method, and connects a terminal of each conducting wire of the plurality of electrode patches with a coaxial signal plug, and then connects the coaxial signal plug into an insert hole disposed on a lateral side of the casing.

5. The display storage apparatus capable of detecting a pulse as recited in claim 1 wherein the heartbeat detector unit sends a measured heartbeat signal back to the casing by a wireless transmission method, and connects a terminal of each conducting wire of the plurality of electrode patches with a wireless signal transmitter circuit for transmitting the signal, and then a wireless signal receiver circuit installed in the casing receives the signal.

6. The display storage apparatus capable of detecting a pulse as recited in claim 1 wherein the plurality of electrode patches includes an anode patch, a cathode patch and a common electrode patch, all made of a metal of a metal sheet or an electrically conducting rubber, and a contact surface of the electrode patches with a human chest is adhesive.

7. The display storage apparatus capable of detecting a pulse as recited in claim 1 wherein the USB interface is in a form of a male connector and convexly disposed on a lateral side of the casing.

8. The display storage apparatus capable of detecting a pulse as recited in claim 1 wherein the USB interface is in a form of a female connector and concavely disposed on a lateral side of the casing.

9. The display storage apparatus capable of detecting a pulse as recited in claim 1 wherein the power supply unit includes a battery.
